(19) United States
(12) Patent Application Publication
(10) Pub. No.: US 2018/0271919 A1
(43) Pub. Date: Sep. 27, 2018

(54) PROBIOTIC COMPOSITION AT LEAST COMPRISING BIFIDOBACTERIUM BIFIDUM W23 AND CAPABLE OF CONTROLLING INTESTINAL BARRIER FUNCTION

(71) Applicant: Winclove Holding B.V., Amsterdam (NL)

(72) Inventor: Saskia van Hemert, Lelystad (NL)

(21) Appl. No.: 15/548,499

(22) PCT Filed: Feb. 3, 2016
(86) PCT No.: PCT/EP2016/052285

§ 371 Date: Aug. 3, 2017

(30) Foreign Application Priority Data
Feb. 3, 2015 (EP) 15153724.8

(51) Int. Cl. A61K 35/747; A61K 35/745; A61K 35/744

(52) U.S. Cl. CPC A61K 35/747; A61K 35/744; A61K 35/745

(57) ABSTRACT

The disclosure relates to the field of medicine and nutrition, more specifically, to the field of treatment and prevention of human disorders such as depression, rumination, aggression, migraine, autistic spectrum disorders (including autism and ADHD), schizophrenia, chronic fatigue, kidney disorders, metabolic syndrome or diabetes type II. The disclosure provides a pharmaceutical or food composition or a supplement comprising a multispecies probiotic composition at least comprising Bifidobacterium bifidum W23 for use in the treatment or prevention of disorders in humans and methods to use such a pharmaceutical or food composition or a supplement.

### CROSS-REFERENCE TO RELATED APPLICATIONS

This application is a national phase entry under 35 U.S.C. § 371 of International Patent Application PCT/EP2016/052285, filed Feb. 3, 2016, designating the United States of America and published in English as International Patent Publication WO 2016/124642 A1 on Aug. 11, 2016, which claims the benefit under Article 8 of the Patent Cooperation Treaty to European Patent Application Serial No. 15153724.8, filed Feb. 3, 2015.

### TECHNICAL FIELD

This application relates to the field of medicine and nutrition, more specifically to the field of treatment and prevention of human disorders involving the brain-gut axis, such as depression, rumination, aggression, migraine, autistic spectrum disorders (including autism and ADHD), schizophrenia, chronic fatigue, and kidney disorders, metabolic syndrome or diabetes type II.

### BACKGROUND

The intestine and the brain are intimately connected via the brain-gut axis, which involves bidirectional communication via neural, endocrine and immune pathways. In recent years it has become increasingly evident that this communication also involves interactions with the intestinal microbiota, which release immune activating and other signaling molecules that may play an important role in regulating the brain and subsequent behavior. For example, the microbiota produce neuroactive substances and their precursors (e.g., tryptophan), which can reach the brain via endocrine and afferent autonomic pathways. Also, bacterial products, such as the gram-negative endotoxins, can influence mood and cognitive functions via indirect (e.g., immune activation) and direct (e.g., toll-like receptors on glial cells) mechanisms. These novel insights have fueled the hypothesis that modification of microbial ecology, for example, by supplements containing microbial species (probiotics), may be used therapeutically to modify stress responses and symptoms of anxiety and depression. While most of this research is relatively recent, and predominantly involves animal and pre-clinical human studies, the results appear in support of this hypothesis. For instance, some observed a reduction in anxious and depressive behavior after feeding healthy mice with Lactobacillus rhamnosus JB-1, or a reduction in depressive-like behaviors in adult rats after feeding them with Bifidobacterium infantis 35624. This reduction was comparable to the effects of administering the antidepressant citalopram. Probiotic studies in humans are still scarce, but the available data are promising. It was found in a non-clinical sample that a three-week intervention with a probiotics-containing milk drink (i.e., Lactobacillus casei Shirota) improved mood scores compared to participants who received a placebo intervention. Improvement in mood was only observed for participants who showed elevated symptoms of depression at baseline. In another pre-clinical study it was demonstrated that participants who were given a mixture of probiotics containing Lactobacillus helveticus R0052 and Bifidobacterium longum R0175 showed significantly less psychological distress than matched controls. Furthermore, it was demonstrated that patients with chronic fatigue syndrome, which is often comorbid with anxiety disorders, reported significantly less anxiety symptoms after ingestion of a daily dose of L. casei Shirota for two months, as compared to a placebo group. On the basis of these and other results it has been suggested that probiotics may serve as adjuvant or preventive therapy for depression.

Given the anti-inflammatory capacity of probiotics and the increasing evidence that there may be a role for microbiota in the brain-gut axis communication, probiotics may have a role in improving physical, mental and social function, reducing depression and anxiety. The intestinal epithelium is a single cell layer that forms the largest and most important barrier against the external environment. A proper functioning of the intestinal barrier is essential for maintaining optimal health. A disrupted intestinal barrier function has been associated with the development of autoimmune and inflammatory diseases. Probiotics are live microorganisms that confer a health benefit to the host and have been associated with prevention of diseases such as antibiotic-associated diarrhea, irritable bowel syndrome and inflammatory bowel disease. Probiotics can improve and restore the intestinal barrier function in diverse ways and effects are thought to be species- and even strain-specific. New studies indicate that gut microbiota communicates with the central nervous system, and thus influences brain function and behavior including anxiety and depression (Steenbergen et al., Brain, Behavior, and Immunity (2015) 48:258-264). Furthermore, the incidence of obesity, metabolic syndrome and type 2 diabetes is growing. The intestinal microbiota and the epithelial barrier function might play an important role in these conditions. For example, the development of type 2 Diabetes Mellitus (T2DM) is a multifactorial process: lifestyle, eating habits, genetics all play an important role. It is expected that in The Netherlands in 2030 over one million people will be diagnosed with T2DM (Diabetes Fund). One of the important risk factors is obesity, being overweight or obese causes 40% of the new T2DM patients. The relation between overweight and diabetes is complex, cause and effect are not known yet. Both T2DM and obesity are associated with increased intestinal permeability and mild chronic inflammation. Increased intestinal permeability may be caused by a disrupted intestinal microbiota composition. Also, several studies demonstrated significant associations between migraine and celiac disease, inflammatory bowel disease, and IBS. Possible underlying mechanisms of migraine and GI diseases could be increased gut permeability and inflammation. Therefore, it would be worthwhile to investigate these mechanisms further in migraine patients. These mechanisms also give a rationale to investigate the effects of the use of pre- and probiotics in migraine patients. The same may also be said for seemingly related mental disorders such as autism and schizophrenia, and kidney disorders.

Depression is a disorder that currently affects about 350 million people worldwide, with mental healthcare costs estimated to be billions of dollars, and with fewer than half of those affected receiving treatment. The increasing incidence of depression in the population is so alarming that developing preventive measures should be a priority. Among the factors that predict the onset of (and vulnerability to) depression, cognitive reactivity seems to be particularly important. Cognitive reactivity refers to dysfunctional cognitive thought patterns that emerge as a result of a sad mood, and is considered one of the most important vulnerability markers of depression, especially for ruminative thoughts and aggression. Interestingly, cognitive reactivity to sad mood seems to be associated with serotonin concentrations, with higher scores reflecting lower serotonin levels. Given the relationship between cognitive reactivity and serotonin on the one hand, and between probiotics and serotonin on the other, it is reasonable to search for probiotics intervention to affect cognitive reactivity such as sad mood.

Some suggest that some probiotics have a therapeutic potential in mood and anxiety disorders, and that they may serve as adjuvant therapy for depression. Chronic inflammatory diseases (autoimmunity, allergy and inflammatory bowel diseases) are increasing in prevalence in urban communities in high-income countries. One important factor is reduced exposure to immunoregulation-inducing microorganisms and microbiota that accompanied mammalian evolution (the hygiene hypothesis or “Old Friends” mechanism). Reduced exposure to these organisms predisposes to poor regulation of inflammation. But inflammation may be equally relevant to psychiatric disorders. Inflammatory mediators may modulate brain development, cognition and mood, and accompany low socioeconomic status and some cases of depression in developed countries. The risk of all these conditions (chronic inflammatory and psychiatric) is increased in urban versus rural communities. Diminished exposure to immunoregulation-inducing Old Friends in the perinatal period may enhance the consequences of psychosocial stressors, which induce increased levels of inflammatory mediators, modulate the microbiota and increase the risk for developing all known psychiatric conditions. In later life, the detrimental effects of psychosocial stressors may be exaggerated when the stress occurs against a background of reduced immunoregulation, so that more inflammation (and, therefore, more psychiatric symptoms) result from any given level of psychosocial stress. This interaction between immunoregulatory deficits and psychosocial stressors may lead to reduced stress resilience in modern urban communities. This concept suggests novel interpretations of recent epidemiology, and novel approaches to the increasing burden of psychiatric disease. Indeed, the intestinal microbiota is the largest source of microbial stimulation that exerts both harmful and beneficial effects on human health. However, more detailed studies are needed to determine the precise action mode of probiotics on both mucosal and systemic immunity. The interaction between probiotic and enterocytes is the initiating event in immunomodulation and merits particular attention. The effects of probiotics are strain dependent and for each new probiotic strain, profiles of cytokines secreted by lymphocytes, enterocytes or dendritic cells that come in contact with the strain may differ. To evaluate the effects of probiotics on the immune system, experimental animal models that mimic the mucosa, and thus a physiological reality, should be preferred whenever it is possible. However, observed effects of strains in experimental animals cannot be directly translated to humans but should be backed up by properly conducted randomized double bind clinical studies. Typically, one differential characteristic of some strains of bacteria that are used as probiotics compared with other microorganisms is their ability to survive during gastrointestinal transit. This allows them to interact with commensal microbiota and/or intestinal epithelial cells and also with mucosa-associated lymphoid cells. The communications between these systems result in the induction or modulation of a number of biological activities that may or may not provide beneficial effects for health. It is accepted that not all strains show probiotic effects always and in all species, and that, with respect to their effects on the host's immune system, a high variability among species and among different strains of the same species is to be expected. A common misconception is that probiotics must always colonize the intestinal tract to exert their effects. In fact, some probiotics become part of the human intestinal microbiota, whereas others may not. Non-colonizing probiotics must then indirectly exert their effects either in a transient manner as they pass through or, more likely, by remodeling or influencing the existing microbial community. The intestinal barrier is important in preventing translocation of bacteria, toxins and antigens from the lumen of the gut into the body. Enhanced permeability, or gut leakiness, has been associated with different diseases. Probiotics can, strain-specifically, improve the epithelial barrier function. However, so far, most researchers have used cell lines or animal models due to the difficulty of measuring the effects of products on the epithelial barrier function in vivo in humans. Recently, a systematic literature search was performed to find articles addressing the effects of probiotics on the barrier function in human trials (Advances in Microbiology, 2013, 3:212-221). The Pubmed database was searched (January 2013) to identify human in vivo studies with probiotic products in which parameters for epithelial barrier function were measured. In total, 29 studies were identified, but patients, bacterial characteristics and methods to measure intestinal barrier function caused large heterogeneity among these studies. About half of the studies showed positive results of probiotics on the epithelial barrier function, indicating a clear potential of probiotics in this field. In a case series of fourteen patients using ECOLOGIC® 825, a probiotic food supplement with known effect on epithelial barrier function, different markers of intestinal integrity improved significantly. Further studies in this field should consider strain(s), dose and duration of the probiotic supplementation as well as the markers used to measure epithelial barrier function.

### BRIEF SUMMARY

The disclosure provides, among other things, a supplement comprising a multispecies probiotic composition at least comprising or containing Bifidobacterium bifidum W23 for use in the treatment or prevention of gut-brain disorders (including depression, migraine, autistic spectrum disorders, or schizophrenia) in humans. The disclosure provides, among other things, results obtained by assessing the possible beneficial effect of probiotics on brain-gut axis by testing on cognitive reactivities to sad mood, a vulnerability marker for depression. To this end, healthy individuals without any current mood disorder underwent a four-week intervention period, during which they were supplied with either probiotics or an inert placebo. The effect of multispecies probiotics containing different strains and species of the genera Lactobacillus, Lactococcus and Bifidobacterium was tested (see below for further details). Some of these genera have been found to be effective in ameliorating anxious and depressive symptoms, however, importantly, studies have shown that multispecies probiotics (i.e., combining different strains of specific genera) can have increased effectiveness through an additive effect of specific strain properties such as colonization of different niches, enhanced adhesion and induction of an optimal pH range, as compared to monospecies supplements. However, some probiotics may compete with each other in terms of functionality and, therefore, the assumption that combinations of different strains may have additive effects needs verification on a preparation-by-preparation basis. The disclosure provides a supplement comprising a multispecies probiotic composition at least comprising or containing Bifidobacterium bifidum W23 for use in the treatment or prevention of depression or aggression or rumination in humans. The multispecies probiotic composition may additionally be provided with tryptophan. This strain, together with Lactobacillus acidophilus W37 and Lactobacillus brevis W63, was shown to generate the greatest contribution to protect an epithelial cell against cytokine-induced dysfunction and was also shown to protect against most parameters affecting barrier function tested (see also Table 2). It is preferred that the pharmaceutical or food composition or a supplement additionally comprises or contains Lactobacillus acidophilus W37 and/or Lactobacillus brevis W63. In a further preferred embodiment, the disclosure provides a supplement according to the disclosure additionally comprising or containing Lactobacillus casei W56 and/or Lactobacillus salivarius W24, and/or additionally comprising or containing Bifidobacterium lactis W52 and/or Lactococcus lactis W19 and/or Lactococcus lactis W58 to further the beneficial properties of the probiotic composition. In a preferred embodiment, the disclosure provides a supplement comprising a multispecies probiotic composition with Bifidobacterium bifidum W23 and Lactobacillus acidophilus W37 and Lactobacillus brevis W63 and Lactobacillus casei W56 and Lactobacillus salivarius W24 and Bifidobacterium lactis W52 and Lactococcus lactis W19 and Lactococcus lactis W58. The multispecies probiotic composition may additionally be provided with tryptophan.

The disclosure also provides a pharmaceutical comprising a multispecies probiotic composition at least comprising or containing Bifidobacterium bifidum W23 for use in the treatment or prevention of depression or aggression or rumination in humans. This strain, together with Lactobacillus acidophilus W37 and Lactobacillus brevis W63, was shown to generate the greatest contribution to protect an epithelial cell against cytokine-induced dysfunction and was also shown to protect against most parameters of probiotic function tested (see also Table 2). It is preferred that the pharmaceutical additionally comprises or contains Lactobacillus acidophilus W37 and/or Lactobacillus brevis W63. In a further preferred embodiment, the disclosure provides a pharmaceutical according to the disclosure additionally comprising or containing Lactobacillus casei W56 and/or Lactobacillus salivarius W24, and/or additionally comprising or containing Bifidobacterium lactis W52 and/or Lactococcus lactis W19 and/or Lactococcus lactis W58 to further the beneficial properties of the probiotic composition. In a preferred embodiment, the disclosure provides a pharmaceutical comprising a multispecies probiotic composition with Bifidobacterium bifidum W23 and Lactobacillus acidophilus W37 and Lactobacillus brevis W63 and Lactobacillus casei W56 and Lactobacillus salivarius W24 and Bifidobacterium lactis W52 and Lactococcus lactis W19 and Lactococcus lactis W58.

The disclosure provides a food composition comprising a multispecies probiotic composition at least comprising or containing Bifidobacterium bifidum W23 for use in the treatment or prevention of depression or aggression or rumination in humans. This strain, together with W37 and W63, was shown to generate the greatest contribution to protect an epithelial cell against cytokine induced dysfunction and was also shown to protect against most parameters of probiotic function tested (see also Table 2). It is preferred that the food composition additionally comprises or contains Lactobacillus acidophilus W37 and/or Lactobacillus brevis W63. In a further preferred embodiment, the disclosure provides a food composition according to the disclosure additionally comprising or containing Lactobacillus casei W56 and/or Lactobacillus salivarius W24, and/or additionally comprising or containing Bifidobacterium lactis W52 and/or Lactococcus lactis W19 and/or Lactococcus lactis W58 to further the beneficial properties of the probiotic composition. In a preferred embodiment, the disclosure provides a pharmaceutical or food composition or a supplement comprising a multispecies probiotic composition with Bifidobacterium bifidum W23 and Lactobacillus acidophilus W37 and Lactobacillus brevis W63 and Lactobacillus casei W56 and Lactobacillus salivarius W24 and Bifidobacterium lactis W52 and Lactococcus lactis W19 and Lactococcus lactis W58. In a much-preferred embodiment, the disclosure provides a pharmaceutical or food composition or a supplement according to the disclosure for use in the treatment or prevention of rumination and in the treatment or prevention of aggression. The multispecies probiotic composition may additionally be provided with tryptophan.

The disclosure also provides a composition for use in the treatment or prevention of migraine in humans comprising a multispecies probiotic composition at least comprising or containing Bifidobacterium bifidum W23 for use in the treatment or prevention of depression or aggression or rumination in humans. This strain, together with Lactobacillus acidophilus W37 and Lactobacillus brevis W63, was shown to generate the greatest contribution to protect an epithelial cell against cytokine-induced dysfunction and was also shown to protect against most parameters of probiotic function tested (see also Table 2). It is preferred that the pharmaceutical additionally comprises or contains Lactobacillus acidophilus W37 and/or Lactobacillus brevis W63. In a further preferred embodiment, the disclosure provides a pharmaceutical according to the disclosure additionally comprising or containing Lactobacillus casei W56 and/or Lactobacillus salivarius W24, and/or additionally comprising or containing Bifidobacterium lactis W52 and/or Lactococcus lactis W19 and/or Lactococcus lactis W58 to further the beneficial properties of the probiotic composition. In a preferred embodiment, the disclosure provides a pharmaceutical comprising a multispecies probiotic composition with Bifidobacterium bifidum W23 and Lactobacillus acidophilus W37 and Lactobacillus brevis W63 and Lactobacillus casei W56 and Lactobacillus salivarius W24 and Bifidobacterium lactis W52 and Lactococcus lactis W19 and Lactococcus lactis W58 for use in the treatment or prevention of migraine in humans. The multispecies probiotic composition may additionally be provided with tryptophan.

The disclosure also provides a composition for use in the treatment or prevention of schizophrenia in humans comprising a multispecies probiotic composition at least comprising or containing Bifidobacterium bifidum W23 for use in the treatment or prevention of depression or aggression or rumination in humans. This strain, together with Lactobacillus acidophilus W37 and Lactobacillus brevis W63, was shown to generate the greatest contribution to protect an epithelial cell against cytokine-induced dysfunction and was also shown to protect against most parameters of probiotic function tested (see also Table 2). It is preferred that the pharmaceutical additionally comprises or contains Lactobacillus acidophilus W37 and/or Lactobacillus brevis W63. In a further preferred embodiment, the disclosure provides a pharmaceutical according to the disclosure additionally comprising or containing Lactobacillus casei W56 and/or Lactobacillus salivarius W24, and/or additionally comprising or containing Bifidobacterium lactis W52 and/or Lactococcus lactis W19 and/or Lactococcus lactis W58 to further the beneficial properties of the probiotic composition. In a preferred embodiment, the disclosure provides a pharmaceutical comprising a multispecies probiotic composition with Bifidobacterium bifidum W23 and Lactobacillus acidophilus W37 and Lactobacillus brevis W63 and Lactobacillus casei W56 and Lactobacillus salivarius W24 and Bifidobacterium lactis W52 and Lactococcus lactis W19 and Lactococcus lactis W58 for use in the treatment or prevention of schizophrenia in humans. The multispecies probiotic composition may additionally be provided with tryptophan.

The disclosure also provides a composition for use in the treatment or prevention of autistic spectrum disorders (ASD, including autism and ADHD) in humans comprising a multispecies probiotic composition at least comprising or containing Bifidobacterium bifidum W23 for use in the treatment or prevention of depression or aggression or rumination in humans. This strain, together with Lactobacillus acidophilus W37 and Lactobacillus brevis W63, was shown to generate the greatest contribution to protect an epithelial cell against cytokine-induced dysfunction and was also shown to protect against most parameters of probiotic function tested (see also Table 2). It is preferred that the pharmaceutical additionally comprises or contains Lactobacillus acidophilus W37 and/or Lactobacillus brevis W63. In a further preferred embodiment, the disclosure provides a pharmaceutical according to the disclosure additionally comprising or containing Lactobacillus casei W56 and/or Lactobacillus salivarius W24, and/or additionally comprising or containing Bifidobacterium lactis W52 and/or Lactococcus lactis W19 and/or Lactococcus lactis W58 to further the beneficial properties of the probiotic composition. In a preferred embodiment, the disclosure provides a pharmaceutical comprising a multispecies probiotic composition with Bifidobacterium bifidum W23 and Lactobacillus acidophilus W37 and Lactobacillus brevis W63 and Lactobacillus casei W56 and Lactobacillus salivarius W24 and Bifidobacterium lactis W52 and Lactococcus lactis W19 and Lactococcus lactis W58 for use in the treatment or prevention of autism in humans. The multispecies probiotic composition may additionally be provided with tryptophan.

The disclosure also provides a composition for use in the treatment or prevention of metabolic syndrome in humans comprising a multispecies probiotic composition at least comprising or containing Bifidobacterium bifidum W23 for use in the treatment or prevention of depression or aggression or rumination in humans. This strain, together with Lactobacillus acidophilus W37 and Lactobacillus brevis W63, was shown to generate the greatest contribution to protect an epithelial cell against cytokine-induced dysfunction and was also shown to protect against most parameters of probiotic function tested (see also Table 2). It is preferred that the pharmaceutical additionally comprises or contains Lactobacillus acidophilus W37 and/or Lactobacillus brevis W63. In a further preferred embodiment, the disclosure provides a pharmaceutical according to the disclosure additionally comprising or containing Lactobacillus casei W56 and/or Lactobacillus salivarius W24, and/or additionally comprising or containing Bifidobacterium lactis W52 and/or Lactococcus lactis W19 and/or Lactococcus lactis W58 to further the beneficial properties of the probiotic composition. In a preferred embodiment, the disclosure provides a pharmaceutical comprising a multispecies probiotic composition with Bifidobacterium bifidum W23 and Lactobacillus acidophilus W37 and Lactobacillus brevis W63 and Lactobacillus casei W56 and Lactobacillus salivarius W24 and Bifidobacterium lactis W52 and Lactococcus lactis W19 and Lactococcus lactis W58 for use in the treatment or prevention of metabolic syndrome in humans. The multispecies probiotic composition may additionally be provided with tryptophan.

The disclosure also provides a composition for use in the treatment or prevention of fatigue such as chronic fatigue syndrome (CFS), myalgic encephalomyelitis (ME), or fatigue in kidney transplant patients) in humans comprising a multispecies probiotic composition at least comprising or containing Bifidobacterium bifidum W23 for use in the treatment or prevention of depression or aggression or rumination in humans. This strain, together with Lactobacillus acidophilus W37 and Lactobacillus brevis W63, was shown to generate the greatest contribution to protect an epithelial cell against cytokine-induced dysfunction and was also shown to protect against most parameters of probiotic function tested (see also Table 2). It is preferred that the pharmaceutical additionally comprises or contains Lactobacillus acidophilus W37 and/or Lactobacillus brevis W63. In a further preferred embodiment, the disclosure provides a pharmaceutical according to the disclosure additionally comprising or containing Lactobacillus casei W56 and/or Lactobacillus salivarius W24, and/or additionally comprising or containing Bifidobacterium lactis W52 and/or Lactococcus lactis W19 and/or Lactococcus lactis W58 to further the beneficial properties of the probiotic composition. In a preferred embodiment, the disclosure provides a pharmaceutical comprising a multispecies probiotic composition with Bifidobacterium bifidum W23 and Lactobacillus acidophilus W37 and Lactobacillus brevis W63 and Lactobacillus casei W56 and Lactobacillus salivarius W24 and Bifidobacterium lactis W52 and Lactococcus lactis W19 and Lactococcus lactis W58 for use in the treatment or prevention of fatigue such as CFS, ME, or fatigue in kidney transplant patients in humans. The multispecies probiotic composition may additionally be provided with tryptophan.

The disclosure also provides a composition for use in the treatment or prevention of kidney disorders in humans comprising a multispecies probiotic composition at least comprising or containing Bifidobacterium bifidum W23 for use in the treatment or prevention of depression or aggression or rumination in humans. This strain, together with Lactobacillus acidophilus W37 and Lactobacillus brevis W63, was shown to generate the greatest contribution to protect an epithelial cell against cytokine-induced dysfunction and was also shown to protect against most parameters of probiotic function tested (see also Table 2). It is preferred that the pharmaceutical additionally comprises or contains Lactobacillus acidophilus W37 and/or Lactobacillus brevis W63. In a further preferred embodiment, the disclosure provides a pharmaceutical according to the disclosure additionally comprising or containing Lactobacillus casei W56 and/or Lactobacillus salivarius W24, and/or additionally comprising or containing Bifidobacterium lactis W52 and/or Lactococcus lactis W19 and/or Lactococcus lactis W58 to further the beneficial properties of the probiotic composition. In a preferred embodiment, the disclosure provides a pharmaceutical comprising a multispecies probiotic composition with Bifidobacterium bifidum W23 and Lactobacillus acidophilus W37 and Lactobacillus brevis W63 and Lactobacillus casei W56 and Lactobacillus salivarius W24 and Bifidobacterium lactis W52 and Lactococcus lactis W19 and Lactococcus lactis W58 for use in the treatment or prevention of kidney disorders in humans. The multispecies probiotic composition may additionally be provided with tryptophan.

The disclosure also provides a composition for use in the treatment or prevention of diabetes type II in humans comprising a multispecies probiotic composition at least comprising or containing Bifidobacterium bifidum W23 for use in the treatment or prevention of depression or aggression or rumination in humans. This strain, together with Lactobacillus acidophilus W37 and Lactobacillus brevis W63, was shown to generate the greatest contribution to protect an epithelial cell against cytokine-induced dysfunction and was also shown to protect against most parameters of probiotic function tested (see also Table 2). It is preferred that the pharmaceutical additionally comprises or contains Lactobacillus acidophilus W37 and/or Lactobacillus brevis W63. In a further preferred embodiment, the disclosure provides a pharmaceutical according to the disclosure additionally comprising or containing Lactobacillus casei W56 and/or Lactobacillus salivarius W24, and/or additionally comprising or containing Bifidobacterium lactis W52 and/or Lactococcus lactis W19 and/or Lactococcus lactis W58 to further the beneficial properties of the probiotic composition. In a preferred embodiment, the disclosure provides a pharmaceutical comprising a multispecies probiotic composition with Bifidobacterium bifidum W23 and Lactobacillus acidophilus W37 and Lactobacillus brevis W63 and Lactobacillus casei W56 and Lactobacillus salivarius W24 and Bifidobacterium lactis W52 and Lactococcus lactis W19 and Lactococcus lactis W58 for use in the treatment or prevention of diabetes type II in humans. The multispecies probiotic composition may additionally be provided with tryptophan.

The disclosure also provides a method for treating or preventing depression comprising administering to a human subject a multispecies probiotic composition at least comprising or containing Bifidobacterium bifidum W23. In a preferred embodiment of the disclosure, a method for treating or preventing depression is provided wherein the composition additionally comprises or contains Lactobacillus acidophilus W37 and/or Lactobacillus brevis W63. In a further preferred embodiment, the disclosure provides a method for treating or preventing depression wherein the composition additionally comprises or contains Lactobacillus casei W56 and/or Lactobacillus salivarius W24, and/or Bifidobacterium lactis W52 and/or Lactococcus lactis W19 and/or Lactococcus lactis W58 to further the beneficial properties of the probiotic composition. In a much preferred embodiment, the disclosure provides a method for treating or preventing depression comprising administering to a human subject a multispecies probiotic composition comprising or containing Bifidobacterium bifidum W23 and Lactobacillus acidophilus W37 and Lactobacillus brevis W63 and Lactobacillus casei W56 and Lactobacillus salivarius W24 and Bifidobacterium lactis W52 and Lactococcus lactis W19 and Lactococcus lactis W58 for use in the treatment of depression. The multispecies probiotic composition may additionally be provided with tryptophan. The method for treating or prevention of depression may additionally be used for treatment of any of migraine, metabolic syndrome, diabetes type II, schizophrenia, kidney disorders or autism accompanying the depression.

The disclosure also provides a method for treating or preventing rumination comprising administering to a human subject a multispecies probiotic composition at least comprising or containing Bifidobacterium bifidum W23. In a preferred embodiment of the disclosure, a method for treating or preventing rumination is provided wherein the composition additionally comprises or contains Lactobacillus acidophilus W37 and/or Lactobacillus brevis W63. In a further preferred embodiment, the disclosure provides a method for treating or preventing rumination wherein the composition additionally comprises or contains Lactobacillus casei W56 and/or Lactobacillus salivarius W24, and/or Bifidobacterium lactis W52 and/or Lactococcus lactis W19 and/or Lactococcus lactis W58 to further the beneficial properties of the probiotic composition. In a much preferred embodiment, the disclosure provides a method for treating or preventing rumination comprising administering to a human subject a multispecies probiotic composition comprising or containing Bifidobacterium bifidum W23 and Lactobacillus acidophilus W37 and Lactobacillus brevis W63 and Lactobacillus casei W56 and Lactobacillus salivarius W24 and Bifidobacterium lactis W52 and Lactococcus lactis W19 and Lactococcus lactis W58. The multispecies probiotic composition may additionally be provided with tryptophan. The method for treating or prevention of rumination may additionally be used for treatment of any of migraine, metabolic syndrome and diabetes type II, schizophrenia, kidney disorders or autism accompanying the rumination. The disclosure also provides a method for treating or preventing aggression comprising administering to a human subject a multispecies probiotic composition at least comprising or containing Bifidobacterium bifidum W23. In a preferred embodiment of the disclosure, a method for treating or preventing aggression is provided wherein the composition additionally comprises or contains Lactobacillus acidophilus W37 and/or Lactobacillus brevis W63. In a further preferred embodiment, the disclosure provides a method for treating or preventing aggression wherein the composition additionally comprises or contains Lactobacillus casei W56 and/or Lactobacillus salivarius W24, and/or Bifidobacterium lactis W52 and/or Lactococcus lactis W19 and/or Lactococcus lactis W58 to further the beneficial properties of the probiotic composition. In a much preferred embodiment, the disclosure provides a method for treating or preventing aggression comprising administering to a human subject a multispecies probiotic composition comprising or containing Bifidobacterium bifidum W23 and Lactobacillus acidophilus W37 and Lactobacillus brevis W63 and Lactobacillus casei W56 and Lactobacillus salivarius W24 and Bifidobacterium lactis W52 and Lactococcus lactis W19 and Lactococcus lactis W58. The method for treating or prevention of aggression may additionally be used for treatment of any of migraine, metabolic syndrome and diabetes type II, schizophrenia, kidney disorders or autism accompanying the aggression. In another preferred embodiment, the disclosure provides a pharmaceutical or food composition or a supplement comprising a multispecies probiotic composition with Bifidobacterium bifidum W23 and Lactobacillus acidophilus W37 and Lactobacillus brevis W63 and Lactobacillus casei W56 and Lactobacillus salivarius W24 and Bifidobacterium lactis W52 and Lactococcus lactis W19 and Lactococcus lactis W58, preferably for use in the treatment or prevention of depression in humans. The multispecies probiotic composition may additionally be provided with tryptophan. In another preferred embodiment, the disclosure provides a pharmaceutical or food composition or a supplement comprising a multispecies probiotic composition with Bifidobacterium bifidum W23 and Lactobacillus acidophilus W37 and Lactobacillus brevis W63 and Lactobacillus casei W56 and Lactobacillus salivarius W24 and Bifidobacterium lactis W52 and Lactococcus lactis W19 and Lactococcus lactis W58, preferably for use in the treatment or prevention of rumination in humans. The multispecies probiotic composition may additionally be provided with tryptophan. In another preferred embodiment, the disclosure provides a pharmaceutical or food composition or a supplement comprising a multispecies probiotic composition with Bifidobacterium bifidum W23 and Lactobacillus acidophilus W37 and Lactobacillus brevis W63 and Lactobacillus casei W56 and Lactobacillus salivarius W24 and Bifidobacterium lactis W52 and Lactococcus lactis W19 and Lactococcus lactis W58, preferably for use in the treatment or prevention of aggression in humans. The multispecies probiotic composition may additionally be provided with tryptophan. In another preferred embodiment, the disclosure provides a pharmaceutical or food composition or a supplement comprising a multispecies probiotic composition with Bifidobacterium bifidum W23 and Lactobacillus acidophilus W37 and Lactobacillus brevis W63 and Lactobacillus casei W56 and Lactobacillus salivarius W24 and Bifidobacterium lactis W52 and Lactococcus lactis W19 and Lactococcus lactis W58, preferably for use in the treatment or prevention of migraine in humans. The multispecies probiotic composition may additionally be provided with tryptophan. In another preferred embodiment, the disclosure provides a pharmaceutical or food composition or a supplement comprising a multispecies probiotic composition with Bifidobacterium bifidum W23 and Lactobacillus acidophilus W37 and Lactobacillus brevis W63 and Lactobacillus casei W56 and Lactobacillus salivarius W24 and Bifidobacterium lactis W52 and Lactococcus lactis W19 and Lactococcus lactis W58, preferably for use in the treatment or prevention of schizophrenia in humans. The multispecies probiotic composition may additionally be provided with tryptophan. In another preferred embodiment, the disclosure provides a pharmaceutical or food composition or a supplement comprising a multispecies probiotic composition with Bifidobacterium bifidum W23 and Lactobacillus acidophilus W37 and Lactobacillus brevis W63 and Lactobacillus casei W56 and Lactobacillus salivarius W24 and Bifidobacterium lactis W52 and Lactococcus lactis W19 and Lactococcus lactis W58, preferably for use in the treatment or prevention of autism in humans. The multispecies probiotic composition may additionally be provided with tryptophan. In another preferred embodiment, the disclosure provides a pharmaceutical or food composition or a supplement comprising a multispecies probiotic composition with Bifidobacterium bifidum W23 and Lactobacillus acidophilus W37 and Lactobacillus brevis W63 and Lactobacillus casei W56 and Lactobacillus salivarius W24 and Bifidobacterium lactis W52 and Lactococcus lactis W19 and Lactococcus lactis W58, preferably for use in the treatment or prevention of metabolic syndrome in humans. The multispecies probiotic composition may additionally be provided with tryptophan. In another preferred embodiment, the disclosure provides a pharmaceutical or food composition or a supplement comprising a multispecies probiotic composition with Bifidobacterium bifidum W23 and Lactobacillus acidophilus W37 and Lactobacillus brevis W63 and Lactobacillus casei W56 and Lactobacillus salivarius W24 and Bifidobacterium lactis W52 and Lactococcus lactis W19 and Lactococcus lactis W58, preferably for use in the treatment or prevention of kidney disorders in humans. The multispecies probiotic composition may additionally be provided with tryptophan.

In another preferred embodiment, the disclosure provides a pharmaceutical or food composition or a supplement comprising a multispecies probiotic composition with Bifidobacterium bifidum W23 and Lactobacillus acidophilus W37 and Lactobacillus brevis W63 and Lactobacillus casei W56 and Lactobacillus salivarius W24 and Bifidobacterium lactis W52 and Lactococcus lactis W19 and Lactococcus lactis W58, preferably for use in the treatment or prevention of diabetes type II. The multispecies probiotic composition may additionally be provided with tryptophan.

The disclosure also provides a method for treating or preventing depression comprising administering to a human subject a multispecies probiotic composition as provided herein. The disclosure also provides a method for treating or preventing rumination comprising administering to a human subject a multispecies probiotic composition as provided herein. The disclosure also provides a method for treating or preventing aggression comprising administering to a human subject a multispecies probiotic composition as provided herein. In all methods of the disclosure as provided herein, it is preferred that the human subject has a daily intake of the composition of at least 2×10⁸ cfu, preferably of at least 2×10⁹ cfu. Best results are obtained when treatment is maintained for at least four weeks.

### DETAILED DESCRIPTION

Mental disorders, depression in particular, are becoming a global epidemic. Worldwide, more than 350 million people of all ages suffer from depression. A variety of lifestyle and environmental changes may be responsible for the increased prevalence of mental disorders. One specific area of interest is the relationship between the intestinal microbiota and mental health.

### The Intestinal Microbiota

Whereas the internal milieu of the human body is sterile, virtually every surface area is colonized by micro-organisms. Due to its open connection with the outside world, the gastrointestinal (GI) tract can be regarded as the largest surface area of the human body (200 m²), equating to the size of a tennis court. In addition to its size, it is the area most densely colonized by micro-organisms, in numbers (10¹⁴) that are ten-fold greater than the number of body cells. The complex communities of micro-organisms residing in the GI tract are known as the intestinal microbiota.

The intestinal microbiota plays an important role in numerous aspects of physiology, including the development of immune responses, intestinal barrier homeostasis, the absorption of nutrients and the distribution of somatic and visceral fat. Changes in intestinal microbiota composition, also known as dysbiosis, have been observed in diseases or disorders such as inflammatory bowel disease, irritable bowel syndrome and obesity.

Recently, it has become evident that the microbiota can also influence gut-brain communication, brain function and even behavior. The high co-morbidity between stress-related psychiatric symptoms such as anxiety and various chronic intestinal disorders support a role of the intestinal microbiota in gut-brain axis communication. However, the strongest evidence comes from animal studies. Studies in germ free mice and animals exposed to pathogenic bacterial infections, antibiotics and probiotics indicate a role for the intestinal microbiota in the regulation of anxiety, mood, cognition, pain and behavior.

### The Gut-Brain-Axis

Gut-brain communication, known as the gut-brain-axis, functions bidirectionally. For instance, stress and emotions can influence the microbial composition of the gut through the release of stress hormones or sympathetic neurotransmitters that influence gut physiology (permeability) and alter the microbiota composition. On the other hand, the gut microbiota (and probiotics) can influence cytokine levels, which affect brain function. Multiple direct and indirect mechanisms are involved in this communication that include humeral (cortical, noradrenalin), bacterial (metabolites), immune (cytokines) and neural (vagus nerve and enteric nervous system) pathways. Although studies investigating the role of the intestinal microbiota in the gut-brain-axis predominately involve animal models of behavioral disorders like anxiety, depression and cognitive dysfunction, accumulated evidence suggests that the intestinal microbiota may also play a role in other CNS involved conditions, like pain, autism, multiple sclerosis, migraine and hepatic encephalopathy.

Probiotics are defined by the World Health Organization as “live micro-organisms that, when administered in adequate amounts, confer a health benefit on the host.” Positive factors for probiotic functionality are to survive passage though the gastrointestinal tract (e.g., low gastric pH, bile and digestive enzymes), metabolic activity, adherence to the intestinal mucosa and/or production of antimicrobial substances. Nowadays, multistrain and multispecies probiotic mixtures, which contain more than one probiotic strain, are becoming increasingly popular. On the one hand, compared with single strain probiotics, probiotic mixtures have the possible advantage that the properties of individual strains may have additive or even synergistic effects when put in a mixture. In addition, compared with a single strain probiotic, a wider range of health benefits could be accomplished. On the other hand, combining probiotic strains might also result in reduced efficacy as individual strains may have opposite effects or even inhibit each other. It is, therefore, important to understand the individual contribution of each microbial species in the complex ecosystem present in the human GI tract and carefully select the strains to be combined. A daily intake of minimum 10⁹ to 10¹⁰ cfu/day seems to be required to show an effect.

Modulating microbiota-gut-brain axis communication is emerging as an exciting new concept in health and disease. ECOLOGIC® BARRIER (in literature referred to as ECOLOGIC® BARRIER) is a multispecies probiotic product specifically designed to improve the epithelial barrier and to increase the resistance to disturbances of the intestinal barrier. Impaired barrier function has been shown to be important in different psychiatric diseases. Due to the impaired barrier function, the entrance of endotoxins can be increased, leading to enhanced pro-inflammatory reactions in the brains of susceptible people. The strains in ECOLOGIC® BARRIER have been specifically selected for their capacity to strengthen the epithelial barrier, inhibit pro-inflammatory cytokines and mast cell activation and to decrease lipopolysaccharide load.

ECOLOGIC® Barrier is an established multispecies probiotic composition, which is commercially available at Winclove (on the World Wide Web at winclove.com); Hulstweg 11, 1032 LB, Amsterdam) and also commercially available at Orthica (on the World Wide Web at orthica.nl; Postbus 102951301 AG Almere) under the name Orthiflor Protect.

It contains the following generally available probiotic strains:
Bifidobacterium bifidum W23 (Benef. Microbes. 2010 June; 1(2):121-30).
Bifidobacterium lactis W52 (ibid).
Lactobacillus acidophilus W37 (Am. J Gastroenterol. 2008 January; 103(1):178-89).
Lactobacillus brevis W63 (J. Int. Soc. Sports Nutr. 2012 Sep. 20; 9(1):45).
Lactobacillus casei W56 (J. Bacteriol. 2012 December; 194(23):6638).
Lactobacillus salivarius W24 (Arch. Oral Biol. 2009 February; 54(2):132-7).
Lactococcus lactis W19 (Benef. Microbes. 2011 September; 2(3):183-92).
Lactococcus lactis W58 (Benef. Microbes. 2010 June; 1(2):121-30).

ECOLOGIC® Barrier contains the following other components:
Carrier material: maize starch
Mineral mix
Vegetable protein

A typical total cell count of ECOLOGIC® Barrier packed with matrix in sachets is: 2.5×10⁹ cfu/g and may vary between 1×10⁸ cfu/g and 2.5×10¹⁰ cfu/g, preferably between 2.5×10⁸ cfu/g and 1×10¹⁰ cfu/g. Typically, higher cell counts then 2.5×10⁹ cfu/g, preferably higher than 2.5×10¹⁰ cfu/g, would be employed when the probiotic composition is packed in capsules.

### Recommended Use

Dissolve two grams of ECOLOGIC® Barrier in a glass of water (100 ml), preferably lukewarm. Stir before consumption. Use preferably on an empty stomach, in the morning or before going to sleep. Please note that ECOLOGIC® Barrier may not be suitable for children below the age of one year since these children have not the capacity yet to breakdown D-lactate that is produced by some of the strains present in this product

The individual strains of ECOLOGIC® Barrier are isolated and identified by a routine serial dilution and spread plate method as described, for example, in R. E. Buchanan, N. E. Gibbons: Bergey's Manual for Determinative Bacteriology. The individual strains of ECOLOGIC® Barrier are also identified, for example, by the Dutch independent organization NIZO Food Research, using the following two techniques:
Based on the highest match of a partial DNA sequence of the small subunit (16S) ribosomal RNA gene of this strain with the sequences of the species in the database of the Ribosomal Database Project II (RDP release 9.56),
or
Based on rep-PCR fingerprint profile similarity to a reference culture that was identified as such based on the highest match of a partial DNA sequence of the small subunit (16S) Ribosomal RNA gene of this strain with the sequence of the species in the database of the Ribosomal Database Project II (RDP release 9.56).

The QPS (Qualified Presumption of Safety) approach is a system similar in concept and purpose as the GRAS (Generally Recognized As Safe) definition used in the USA, but modified to take account of the different regulatory practices in Europe. It represents a route to harmonization of approaches for the safety assessment of micro-organisms used in feed/food production and would ensure a better use of assessment resources by focusing on those organisms which represent the greatest risks or uncertainties.

All strains present in ECOLOGIC® Barrier have the QPS-status. Note that the B. lactis as named in the literature is, due to new nomenclature conventions, renamed to B. animalis subspecies lactis. ESFA uses this new nomenclature. Please note that B. lactis is still a valid name to use.

An important safety aspect of probiotics is their susceptibility to antibiotics. The sensitivity or resistance to several antibiotics was tested for bacterial strains in ECOLOGIC® Barrier. A minimal inhibitory concentration (MIC) test was performed by micro bouillon dilution. The MICS for all the strains in ECOLOGIC® Barrier fall below the breakpoints set by the EFSA, with the exception of B. bifidum W23 to clyndamicin and L. brevis W63 to chloramphenicol, however the MIC value of L. brevis W63 falls within the margin of error of measurement (plus or minus one two-fold dilution).

### Real Time Stability Test

Storage of a product should not harm the effectiveness of a product. Total viable cell count is, in that sense, an important aspect of probiotic products. This total viable cell count shows how much live bacteria per gram of the product are still present. From an effectiveness point of view the stability of this total viable cell count is important. Therefore, by defining shelf-life for probiotic products this is based on the total viable cell counts after different periods of time of storage. When products have comparable total viable cell counts during time, the product is considered to be stable and thereby even after longer storage still effective. The best method to measure the total viable cell count in time is the Real Time Stability (RTS) test. In this RTS test products are stored up to 48 months at 25° C. and 60% relative humidity and on regular times the total cell count of the product is measured.

Powder of ECOLOGIC® Barrier (in bulk) from two different batches was stored at 25° C. for 12 months. Analysis of the total viable cell count was performed at T=0, 3, 6, and 12 months. No significant differences were observed.

### Probiotics or Supplements Additionally Comprising Tryptophan (TRP)

Earlier, TRP and probiotics each have been considered not so much as an enhancer of healthy cognitive functioning but rather as a means to reduce the negative side effects of serotonin-related pathologies, such as seasonal affective disorders, mood disorders, sleep disorders, depression and bulimia. The outcomes were mixed: some patients reported significant improvements, while other did not. In healthy individuals, studies have demonstrated that the administration of TRP via a food supplement increases social cooperative behavior and mood, the latter one in a sample of elderly individuals. In addition, it can now be demonstrated that a diet with probiotics modulates the cognitive response to sad mood (Steenbergen, Sellaro, van Hemert, Bosch, and Colzato, 2015). Finally, in an unpublished naturalistic pilot study on 292 individuals, it can be demonstrated that participants with a TRP-rich nutrition regime show better social cognitive performance as measured by the Reading the Mind in the Eyes task (RMET). Given that 5-HT level seems to vary between individuals in a stable and enduring fashion and that aging-related losses in neurochemical and structural brain resources modulate the extent to which common genetic variations affect social cognitive functioning, the function relating brain resources to social cognitive performance may not be nonlinear. The disclosure, therefore, also provides TRP enriched probiotics for use in the treatment of depression, rumination, aggression, nervous disorders and mental disorders related to metabolic syndrome, such as reduced social cognition and reduced emotional processing. Typically, TRP is added to such probiotic or supplement products as provided herein at 5-500 mg/g product (final weight), preferably, at 5-250 mg/g, more preferably at 10-100 mg/g, most preferably at 30-70 mg/g.

### Background of the Product

The intestinal mucosa is the largest interface between the outside world and the human internal milieu. Across a surface area that approximates the size of a soccer field, it is here that the highest concentration of bacteria is prevented from invading our internal environment while allowing nutrient and water absorption by a single cell layer of epithelium. The ability to control the invasion of harmful content from the lumen is called intestinal mucosal barrier function. While the epithelial layer forms the most obvious physical boundary between the inner and outer environment, the full complexity of factors that control intestinal barrier function reaches beyond the epithelium and is not fully understood. Throughout the intestine a single layer of epithelial cells covers the inner surface and is responsible for this barrier function.

Tight junctions are protein structures that allow selective passage of ions and small molecules, but form, in healthy subjects, a tight barrier to protein seized molecules and bacteria. To make matters more complicated, the task of the epithelium is not only to keep bacteria and antigens out while absorbing nutrients, but also to allow contact between luminal contents and immune cells. This occurs through limited and highly controlled uptake of antigen and bacteria. This seemingly paradoxical task is, however, crucial in the induction of targeted and protective mucosal immune responses to pathogens as well as to the development of oral tolerance to commensals and food antigens.

The barrier function of the intestine (before described as actions on level 2) can be influenced by different factors, like heredity, bacterial flora, diet, psychological stress, oxidative stress, exercise, and drugs. Increased permeability of the epithelial barrier has been associated with many gastrointestinal inflammatory disorders, like inflammatory bowel diseases (Crohn's disease, ulcerative colitis and pouchitis), celiac disease. An increased permeability can also lead to increased levels of endotoxins in the blood, which are linked to systemic inflammatory diseases, like metabolic syndrome, diabetes, atherosclerosis, chronic fatigue syndrome, autism, migraine and rheumatoid arthritis. Probiotics have proven capabilities to enhance the epithelial barrier, due to different working mechanisms.

### Development of ECOLOGIC® Barrier

ECOLOGIC® Barrier is specially designed to improve the epithelial barrier and to increase the resistance to disturbances of the intestinal barrier. The probiotic strains were selected based on the following criteria:
In vitro strengthening of the epithelial barrier
Inhibition of mast cell activation
Inhibition of pro-inflammatory cytokines
Decreasing lipopolysaccharide load

### Survival Test

The gastro-intestinal survival model is developed by Winclove Bio Industries in cooperation with the University of Maastricht. The model is an in vitro simulation of the gastrointestinal tract and can be used to measure the survival of probiotic products and strains.

In the model, the pH and the addition of pepsin, pancreatin and bile are regulated to simulate the gastro-intestinal tract.

The total cell count is being measured at four different times: after rehydration (T=0), after stomach simulation (T=1¼ hours), after addition of bile and pancreatin (T=3 hours) and at the end of the test (T=6 hours). The experiment is executed at 37° C. It was found that throughout the gastro-intestinal tract the amount of living cells (CFU/gram as given at the y-axis) stays relatively stable. A decrease of one log is considered normal for probiotic products. ECOLOGIC® Barrier decreases less than one log CFU in this in vitro test and thus it can be concluded that ECOLOGIC® Barrier displays good survival of the gastro-intestinal tract.

### Activity Test

Metabolic or biological activity is one of the most important parameters for the quality of a probiotic product. It is even more important than the total amount of colony-forming units (CFUs) in the product. Due to a certain treatment or addition of a new ingredient to the product, the bacterial cells may be damaged in a way they still survive, but no longer reach their full activity level. These cells will be counted in a viable cell count, but do not have much value for the product. The production of lactic acid is a probiotic effect (a health promoting factor of probiotics), which can be used as an indicator for the metabolic activity of probiotic bacteria. It can be stated that the more lactic acid probiotic bacteria produce, the more metabolic active they are. Therefore, measuring acid production is considered a good method for measuring the activity of probiotic strains.

The production of acids by the probiotic bacteria is being measured during time, after a simulation of passage through the stomach by an acid drop, and is used as a parameter of metabolic activity. ECOLOGIC® Barrier shows good metabolic activity.

### Strengthening of the Epithelial Barrier

As stated in the background information: The ability to control the invasion of harmful content from the lumen is called intestinal mucosal barrier function. The integrity of this barrier function can be measured with transepithelial electrical resistance (TEER). TEER is an in vitro measurement of the movement of ions across the paracellular pathway. Maintenance of the intestinal integrity is critical for essential physiological processes. Therefore, a reduction in TEER may represent an early expression of cell damage and indicates that the barrier function of the intestine is decreased.

A monolayer of villus-like Caco-2 cells was grown on a filter. The experiment was performed with an inflammatory stressor (a combination of TNF-α and IL1-β), which is known to reduce the relative TEER of the Caco-2 cells.

The monolayer of Caco-2 cells was first exposed for 12 hours to the probiotic bacteria followed by exposure to inflammatory stressor in the presence of the same probiotic bacteria, also for 1 hour. After a recovery time of 4 and 24 hours, the TEER of the monolayer was measured. The results were compared to the TEER of a monolayer that was exposed to stressor alone and an unexposed stressor. Three strains (B. bifidum W23, L. acidophilus W37 and L. brevis W63) in ECOLOGIC® Barrier could for more than 90% protect the epithelial cell against the cytokine induced dysfunction of the barrier, whereas two others (L. casei W56 and Lc. lactis W19) had a partial effect, three strains (B. lactis W52, L. salivarius W24 and Lc. lactis W58) did show little effect in this assay. A smaller TEER screening was performed with the pathogenic bacteria Salmonella enteritis 857, which has been shown to decrease the relative TEER in Caco-2 cells. In a small experiment with six bacterial strains, an epithelial cell-line (CaCo-2) was damaged by a pathogenic bacterium, Salmonella enteritidis. Three (W52, W56, and W58) of the six strains were able to diminish the decrease in transepithelial resistance (and thus strengthen the barrier function) due to the Salmonella significantly.

Probiotic bacteria can have an effect on the epithelial barrier via different molecular pathways and routes. Therefore, a multispecies product is a very good choice to support these different routes. All the strains present in ECOLOGIC® Barrier were carefully selected based on their in vitro capacities to improve the epithelial barrier and interact with the immune system in a regulatory manner.

### Vivo Results

### Depression

In the past years, the existence of a gut-brain axis has become clear. This has raised the question whether probiotics can influence brain health. Recently two studies with ECOLOGIC® Barrier have been finished which indicate a positive effect on depression related behaviors.

In the first study, performed at Aarhus University, Denmark, healthy rats were given ECOLOGIC® Barrier or placebo for eight weeks. After that period the animals did a forced swim test, a typical screening for depressive-like behavior in rodents. The more depressive-like behavior they have, the less they move. The group of animals, that used probiotics, moved significantly more compared to the placebo group. However, as discussed before, results in experimental animals such as rodents do not predict results in humans. Therefore, a second study was a human study, performed at Leiden University, The Netherlands. In this randomized, placebo control study, 40 students were taking ECOLOGIC® Barrier (2 grams, 5×10⁹ cfu/day) or placebo for four weeks. Before and after the intervention a validated questionnaire was filled in, the Leiden Index of Depression Sensitivity, Revised. This questionnaire measures cognitive reactivity in response to low mood. At baseline there were no differences between the two groups, but after 4 weeks the score was decreased significantly in the probiotic group compared with the placebo group. The most pronounced decreases were in the categories Aggression (e.g., “When I feel down, I lose my temper more easily”) and Rumination (e.g., “When I feel sad, I spend more time thinking about the possible causes of my moods”).

Probiotics supplementation has been suggested to ameliorate depressive symptoms by increasing levels of tryptophan, the precursor of serotonin. Importantly, lower serotoninergic levels are also associated with cognitive reactivity, that is, the tendency to think negatively when experiencing a sad mood—a marker of depression vulnerability. This suggests that an intervention using probiotics may reduce the cognitive reactivity to sad mood (i.e., vulnerability to depression) by acting on serotonin production in the brain. In a triple-blind, placebo-controlled, randomized, pre- and post-intervention assessment design, twenty participants received a four-week probiotic food-supplement intervention with the multispecies probiotic ECOLOGIC® Barrier while twenty other participants received an inert placebo for the same period of time. In the pre- and post-intervention assessment, cognitive reactivity to sad mood was assessed using the revised Leiden Index of Depression Sensitivity (LEIDS-R) scale. When compared to participants that received the placebo intervention, participants that received the four-week intervention with the probiotic showed significantly reduced overall cognitive reactivity to the sad mood and, specifically, ruminative and aggressive thoughts. These results suggest that the intake of selected probiotics may represent an effective preventive tool for depression-related disorders as it may help overcome negative thoughts associated with sad mood, thus decreasing vulnerability to depression.

A healthy lifestyle, accompanied by eating healthy foods, has become very important for many people over the last years. This is a result of people becoming more and more aware of what a healthy lifestyle can do in terms of long-term health. Research has shown that food supplements not only affect physical functioning, but also social behavior and cognitive functioning. For example, tryptophan—the precursor of serotonin—has been found to increase interpersonal trust, while tyrosine—the precursor of dopamine—has been shown to improve different facets of cognitive control. The idea that the food one eats has a bearing on one's state of mind is further supported by the fact that the intestine and the brain are intimately connected via the brain-gut axis, which allows communication between the gastrointestinal tract and the central nervous system. The gastrointestinal tract includes intestinal microbiota that play an important role in healthy functioning of the brain and subsequent behavior. Even though the exact mechanism remains unclear, intestinal microbiota seem to increase plasma tryptophan levels, thus altering serotonin turnover in the brain. Serotonin is a neurotransmitter that plays a critical role in anxiety and depression disorders. Animal studies have suggested that most of the gut-to-brain signals are transmitted via the vagus nerve. Interestingly, in humans the vagus nerve reaches, via the locus coeruleus and the raphe nuclei (the principal sources of serotonin release in the brain), the anterior cingulate cortex (ACC) and the prefrontal cortex (PFC), in particular, the medial prefrontal cortex (mPFC)—one of the brain regions associated with processing of affect and social information. Consistent with that, studies addressing the role of gastrointestinal microbiota have suggested that these bacterial cells, as well as food supplements containing them (i.e., probiotics), may play a role in modulating the stress response and stress-related behaviors associated with anxiety and depression. For instance, anxious and depressive behavior decreased after feeding healthy mice Lactobacillus rhamnosus JB-1. Similarly, a reduction in symptoms associated with depression was observed in adult rats after feeding them with Bifidobacterium infantis 35624, a reduction comparable to the one induced by the antidepressant citalopram. Studies on the effects of probiotic treatment on humans are still scarce, but encouraging. Lactobacillus helveticus CM4 was demonstrated to improve sleep in elderly subjects. A three-week intervention with a probiotics-containing milk drink (i.e., Lactobacillus casei Shirota) improved mood scores compared to participants who received a placebo intervention. Interestingly, the improvement in mood scores was only observed for participants who truly felt depressed. Along the same line, responding patients with chronic fatigue syndrome, often comorbid with anxiety disorders, who ingested a daily dose of Lactobacillus casei Shirota for two months, showed significantly less anxiety symptoms after the intervention than a placebo group. In another study, it was demonstrated that subjects who were given a mixture of probiotics containing Lactobacillus helveticus R0052 and Bifidobacterium longum R0175 showed significantly less psychological distress than matched controls.

The present study was aimed at assessing the possible beneficial effect of probiotics on cognitive reactivity to sad mood (i.e., vulnerability to depression). To this end, healthy people with no current symptoms of depression or anxiety underwent a four-week intervention period, during which they were supplied with either probiotics or an inert placebo. The effect of multispecies probiotics (containing strains of different probiotic species) was tested, as they seem to be more effective in terms of health benefits than monostrain probiotics (containing one strain of a certain species) and multistrain probiotics (containing more than one strain of the same species. Before and after the intervention, cognitive reactivity was measured by means of the revised Leiden Index of Depression Sensitivity (LEIDS-R), a self-report questionnaire that has been shown to be predictive of depression across multiple longitudinal studies.

We assessed probiotics intervention to help people overcome negative thoughts associated with sad mood, thus decreasing cognitive reactivity. Furthermore, before and after the intervention, participants' levels of depression and anxiety were assessed and controlled for possible changes in self-report measures of depressive and anxiety symptoms across the two sessions. Symptoms of depression were measured by means of the Beck Depression Inventory (BDI-II), whereas symptoms of anxiety were evaluated with the Beck Anxiety Inventory (BAI).

### Material and Methods

### Participants

Forty participants with no cardiac, renal, heptic, psychiatric or neurological disorders, no personal or family history of depression or migraine, no medication or drug use and no allergies or intolerance to lactose or gluten participated in this experiment. Participants were screened via a phone call by the experiment leader before inclusion, using the Mini International Neuropsychiatric Interview (M.I.N.I.). The M.I.N.I. is a short, structured, interview of about 15 minutes that screens for several psychiatric disorders and drug use, often used in clinical and stress research. Participants were equally and randomly assigned to receive a four-week intervention of either placebo or probiotics. Twenty participants (three male) with a mean age of 19.7 years (SD=1.7) and a mean body mass index (BMI) of 21.5 (SD=2.0) were assigned to the placebo condition, and twenty participants (five male) with a mean age of 20.2 years (SD=2.4) and a mean BMI of 22.6 (SD=2.2) were assigned to the probiotics condition. Written informed consent was obtained from all participants and the protocol was approved by the local ethical committee (Leiden University, Institute for Psychological Research).

### Design and Procedure

A blind, at three levels (group allocator, participants, outcome assessor), placebo-controlled, randomized, pre- and post-intervention assessment design, was used to investigate the effect of multispecies probiotic intervention on depression and anxiety, as well as on cognitive reactivity to sad mood (vulnerability marker of depression), in healthy young students. Participants received a four-week food supplementation intervention of either placebo or probiotics. In the probiotics intervention participants were provided with 28 sachets (one for each day of intervention), each containing 2 g freeze-dried powder of the probiotic mixture ECOLOGIC® Barrier (2.5×10⁹ cfu/gram, Winclove probiotics, The Netherlands). ECOLOGIC® Barrier contains a matrix and eight bacterial strains: Bifidobacterium bifidum W23, Bifidobacterium lactis W52, Lactobacillus acidophilus W37, Lactobacillus brevis W63, Lactobacillus casei W56, Lactobacillus salivarius W24, and Lactococcus lactis W19 and W58. The matrix consisted of cornstarch, maltodextrin, vegetable protein, MgSO₄, MnSO₄ and KCl. In the placebo intervention, participants were provided with 28 sachets, each containing 2 g freeze-dried powder of the matrix only. The placebo was indistinguishable from the probiotics sachets in color, taste, and smell, but contained no bacteria.

In the pre- and post-test assessment (session 1 and session 2, respectively), participants were required to fill in the following questionnaires, presented in a fixed order: the revised Leiden Index of Depression Sensitivity (LEIDS-R), the Beck Depression Inventory (BDI-II) [43] and the Beck Anxiety Inventory (BAI). E-prime 2.0 software system (Psychology Software Tools, Inc., Pittsburgh, Pa.) was used to present the questionnaires and to collect participants' responses, which were to be given using a computer. Due to technical problems, one participant assigned to the placebo group did not fill in the pre-intervention BAI questionnaire. After having filled in the three questionnaires, participants were required to perform two social cognitive tasks tapping into reactions to fairness and interpersonal trust. In each session, the complete test battery lasted about 20 minutes.

At the end of session 1 (i.e., pre-intervention assessment) participants were provided with the 28 sachets of powder (containing either the inert placebo or the multispecies probiotics) for the four-week intervention. Participants were instructed to dissolve the powder in water or lukewarm milk and to drink it in the evening before going to bed. Compliance was assured by reminding the participants via a text message sent by the experimenter.

### Questionnaires

The LEIDS-R is a self-report questionnaire with 34 items that assess to what extent dysfunctional thoughts are activated when experiencing mild dysphoria (i.e., it measures cognitive reactivity to sad mood, also referred to as vulnerability to depression). Before answering the items, participants were asked to take a few minutes to imagine how they would feel and think if they were to experience a sad mood and to indicate, on a 5-point Likert scale ranging from 0 (i.e., “not at all”) to 4 (“very strongly”), the extent to which each statement applied to them. It was emphasized that the statements applied to the situations in which they feel sad but not truly down or depressed. The scale consists of six subscales that measure vulnerability with respect to:

Aggression (e.g., When I feel down, I lose my temper more easily);

Hopelessness/Suicidality (e.g., When I feel down, I more often feel hopeless about everything; When I feel sad, I feel more that people would be better off if I were dead);

Acceptance/Coping (e.g., When I am sad, I feel more like myself);

Control/Perfectionism (e.g., I work harder when I feel down);

Risk aversion (e.g., When I feel down, I take fewer risks);

Rumination (e.g., When a feel sad, I more often think about how my life could have been different).

Hopelessness and Acceptance/Coping both consist of five items, with a maximum score of 20 per subscale, whereas the other scales comprise of six items with a maximum score of 24 per subscale. The total LEIDS-R score is given by adding the scores obtained on each subscale, resulting in total scores ranging from 0 to 136. The BDI-II is a 21-item self-report questionnaire that assesses the existence and severity of a current (past two weeks) depression in both adolescents and adults. The Dutch translation as provided by Van der Does was used. The BDI-II has been found to be a valid indicator of depression and showed good diagnostic discrimination in early tests of validity.

Participants were presented with several items related to symptoms of depression and asked to choose for each item the statement that better described how they have been feeling during the past two weeks (including the current day). Items can be grouped in two subscales: affective (i.e., eight items; e.g., hopelessness, irritability, guilt) and somatic/physical (i.e., thirteen items; e.g., fatigue, weight loss, lack of interest in sex). Items are rated on a 4-point scale ranging from 0 to 3 in terms of severity. For the affective subscale, the score ranges from 0 to 24 and for the somatic/physical scale the score ranges from 0 to 39. The total score is given by adding all items, hence, scores range between 0 and 63 (0-13: minimal depression, 14-19: mild depression, 20-28: moderate depression and 29-63: severe depression).

The BAI is a 21-item self-report questionnaire that assesses the existence and the severity of anxiety. The Dutch translation was used. Participants are presented with items describing common symptoms of anxiety (such as numbness and tingling, sweating not due to heat, and fear of the worst happening) and asked to rate, on a 4-point Likert scale (0, not at all, 1, mildly, 2, moderately, 3, severely), how much they have been bothered by each symptom over the past week. Total scores are obtained by summing all items, with values ranging between 0 and 63 (0-7: minimal level of anxiety; 8-15: mild level of anxiety: 16-25: moderate level of anxiety; 26-63: severe level of anxiety).

### Statistical Analyses

For each questionnaire, the mean scores (total and/or partial) were calculated and submitted to a repeated measures analysis of variance (ANOVA) with session (pre- vs. post-intervention) as within-subjects factor and group (placebo vs. probiotics) as between-subjects factor. Newman-Keuls post-hoc analyses were performed to clarify mean differences in case of significant interactions. All alpha levels were set at p=0.05.

In addition to standard statistical methods, Bayesian probabilities associated with the occurrence of the null (p(H₀|D)) and alternative (p(H₁|D)) hypotheses were calculated, given the observed data. This method allows making inferences about both significant and non-significant effects by providing the exact probability of their occurrence. The probabilities range from with 0 (i.e., no evidence) to 1 (i.e., very strong evidence).

### Results

No significant group differences were observed in terms of age, t(38)=−0.76, p=0.45, BMI, t(38)=−1.64, p=0.11 and gender distribution, χ² (1, N=40)=0.63, p=0.43. Table 1 gives a summary of pre- and post-intervention scores on the LEIDS-R, BDI and BAI in the placebo and probiotics groups.

### LEIDS-R

ANOVAs performed on the LEIDS-Rtotal score and on its subscales revealed no main effect of group, Fₛ≤2.93, pₛ≥0.095, pₛ(H₀|D)≥0.59. Significant main effects of session were observed for the LEIDS-R total score, F(1.38)=17.20, p<0.001, ηₚ²=0.312, MSE=40.468, p(H₁|D)≥0.99, aggression, F(1.38)=8.89, p=0.005, ηₚ²=0.190, MSE=4.255, p(H₁|D)=0.91, rumination, F(1.38)=10.62, p=0.002, ηₚ²=0.218, MSE=3.826, p(H₁|D)=0.96, control/perfectionism, F(1.38)=7.76, p=0.008, ηₚ²=0.170, MSE=3.709, p(H₁|D)=0.87), hopelessness, F(1.38)=5.89, p=0.020, ηₚ²=0.134, MSE=2.310, p(H₁|D)=0.74, risk aversion, F(1.38)=4.52, p=0.040, ηₚ²=0.106, MSE=5.851, p(H₁|D)=0.60), but not for acceptance/coping, F<1, p=0.77, p(H₀|D)=0.86. Overall, participants showed lower scores at the post-intervention measurement compared to the pre-intervention measurement. Crucially, significant interactions between session and group were observed for the LEIDS-R total score, F(1.38)=6.05, p=0.019, ηₚ²=0.137, MSE=40.468, p(H₁|D)=0.79, aggression, F(1.38)=4.94, p=0.032, ηₚ²=0.115, MSE=4.255, p(H₁|D)=0.65, and rumination, F(1.38)=12.16, p=0.001, ηₚ²=0.242, MSE=3.826, p(H1|D)=0.98. Post-hoc analyses revealed that participants who received a four-week placebo intervention showed comparable scores in the pre- and post-intervention sessions (total score: 44.70 and 42.30, p=0.465; aggression: 8.80 and 8.45, p=0.85; rumination: 11.75 and 11.85, p=0.87). In contrast, participants who received a four-week probiotics intervention scored significantly lower on the post-intervention compared to the pre-intervention session (total score: 42.75 and 33.35, p<0.001; aggression: 8.65 and 6.25, p=0.002; rumination: 11.20 and 8.25, p<0.001; see Table 1). Thus, these results show that the intake of multispecies probiotics for a four-week period significantly reduced overall cognitive reactivity to depression and, in particular, aggressive and ruminative thoughts.

### BDI

ANOVAs performed on the BDI-II total score and on BDI-II subscales (i.e., affective and somatic) revealed no main effect of session, Fₛ≤0.41, pₛ≥0.52, pₛ(H₀|D)≥0.84, group, Fₛ≤1.08, pₛ≥0.31, pₛ(H₀|D)≥0.78, nor any interaction between the two factors, Fₛ≤0.68, pₛ≥0.52, pₛ(H₀|D)≥0.82. This indicates that the probiotic intervention did not affect scores of self-reported depressive symptoms.

### BAI

No effect of session, F(1,37)=2.30, p=0.14, p(H₀|D)=0.66, group, F(1,37)=0.226, p=0.64, p(H₀|D)=0.85 or interaction, F(1.37)=0.064, p=0.80, pₛ(H₀|D)=0.86, was found, indicating the probiotic intervention did not affect self-reported symptoms of anxiety.

The aim of the current study was to investigate the effect of multispecies probiotic intervention on cognitive reactivity. The probiotic composition as used herein is commercially available under the product name ECOLOGIC® Barrier and is a mixture of bacterial strains (B. bifidum W23, B. Lactis W52, L. acidophilus W37, L. brevis W63, L. casei W56, L. salivarius W24, Lc. Lactis W19, Lc. Lactis W58) in a carrier matrix of maize starch and maltodextrins. More specifically, it was investigated whether supplementation with probiotics can help people overcome dysfunctional cognitive thought patterns, hence, decreasing the vulnerability to depression.

It was found that a four-week multispecies probiotic intervention reduced self-reported cognitive reactivity to depression in general (as indexed by the LEIDS-R). In line with the link between probiotics and serotonin and between lower serotonin levels and higher cognitive reactivity to depression, the observed effect reinforces the idea that probiotics supplementation may act as social and affective cognitive enhancer. This beneficial effect was selective for cognitive reactivity to depression but not self-reported symptoms of depression or anxiety. This is not surprising, given that the sample consisted of healthy individuals with minimal to mild baseline scores on both the BAI and the BDI. More specifically, it was found that probiotics has a beneficial effect on the cognitive reactivity with regard to aggression and rumination subscales, suggesting that a probiotics supplementation of at least four weeks helps people to be less distracted by, and more in control of, aggressive and ruminative thoughts and actions when in a sad mood. It should be noted that these specific effects are very relevant, given that ruminative thoughts, in particular, seem to be a key factor contributing to cognitive reactivity to depression.

Follow-up studies should evaluate the outcome of the probiotics intervention by measuring urine levels of 5-Hydroxyindoleacetic acid (5-HIAA), the main metabolite of serotonin, and by measuring the lactulose/mannitol ratio in urine. The chemical analysis of 5-HIAA in urine samples is commonly used to determine serotonin levels in the body. The lactulose/mannitol test in urine measures intestinal permeability, and an increased intestinal permeability can induce depressive symptoms. Certain probiotics can improve and increase permeability, so this might be a working mechanism involved in the effects of probiotics on cognitive reactivity to depression. Finally, to evaluate the effect of the probiotics intervention on the brain, it would be crucial to measure concentration changes in oxygenated hemoglobin (oxyHb) and deoxygenated hemoglobin (deoxyHb) in the mPFC, a brain region associated with the processing of social and affective information, using for example functional near infrared spectroscopy (fNIRS).

To conclude, it was demonstrated for the first time that a four-week multispecies probiotic intervention has a positive effect on the cognitive reactivity to sad mood (i.e., vulnerability to depression) in healthy individuals that are not currently diagnosed with a depressive disorder. More specifically, the probiotic intervention made participants less vulnerable to aggressive and ruminative thoughts resulting from a sad mood. These findings complement indications from previous studies that probiotics supplementation may have positive effects for depressive patients. However, future studies are needed to assess the neurobiological underpinnings of the brain-gut axis in order to further understand the interactions between brain, probiotics, and behavior. For now, it seems the right bacteria can help us overcome dysfunctional thoughts that may interfere with optimal social and affective functioning.

### Schizophrenia

Schizophrenia is a severe mental disorder with a worldwide prevalence just below 1%, placing a significant burden on global health. Although the introduction of antipsychotic medications in the 1950s has substantially improved clinical symptoms of schizophrenia, the disease is still causing considerable morbidity and mortality. The pathogenesis of schizophrenia is still far from elucidated, which hampers the rational development of novel therapies.

Abnormal immune responses have been reported in patients with schizophrenia, of varying disease stages and medication status. For example, adult schizophrenia patients as a group have elevated serum levels of pro-inflammatory cytokines compared to controls. Symptom severity was found to correlate with levels of inflammatory markers. It has, therefore, been hypothesized that schizophrenia can originate from early exposure to microbial infections, contributing to the etiology through chronic neuroinflammatory and autoimmune processes.

Autoimmunity, atopic disorders, early infection and, more recently, “leaky gut” resulting from gastrointestinal problems, have been associated with schizophrenia for a long time. It is interesting to see that the intestinal microbiota is associated with major psychiatric disorders, including schizophrenia, as this offers a non-invasive and relatively simple strategy to improve symptoms and onset. Potential mechanisms by which the intestinal microbiota affects central nervous system function are immune activation, active neurotransmitters such as GABA, tryptophan metabolism, intestinal hormonal responses and bacterial metabolites. An increased incidence of gastrointestinal barrier dysfunction, food antigen sensitivity, inflammation and the metabolic syndrome are observed in patients with schizophrenia, suggesting a potential deficit in gut microbiota. There are multiple indications that the gastrointestinal barrier is disturbed in schizophrenia patients.

Schizophrenia patients have an increased risk for metabolic syndrome through both intrinsic metabolic abnormalities as well as through the side effects of antipsychotic treatment. Metabolic syndrome is characterized by the presence of three or more specific factors, like elevated waist circumference, elevated triglycerides, reduced HDL cholesterol, elevated blood pressure and elevated fasting glucose. Metabolic syndrome significantly increases the risk for future morbidity and mortality. The overall rate of metabolic syndrome in patients with schizophrenia is 33%, with only minor differences according to treatment setting (inpatient vs. outpatient), country of origin and sex. Antipsychotic medication has been shown to induce weight gain and put patients at a higher risk of the metabolic syndrome. Regarding prescribed antipsychotic medication; highest rates of metabolic syndrome are observed in patients prescribed clozapine (52%) and lowest rates in those who are unmedicated (20%). Antipsychotic-induced weight gain may be mediated through changes in the gastrointestinal tract, which affect systemic inflammation.

The effects of a multispecies probiotic product (ECOLOGIC® Barrier) as compared to a placebo, when given in addition to antipsychotic medication, lowered schizophrenia symptom severity as measured with the PANSS with an average of at least 7.5 points over the course of three months, and provided improvements in cognition in the probiotic over the placebo-treated group. In addition, probiotic treatment decreased presence and severity of metabolic syndrome as well as the gastrointestinal problems and improved immune parameters, which are abnormal in schizophrenia patients.

### Metabolic Syndrome

Metabolic syndrome is defined in different ways by different organizations, but there is agreement on the core components of the metabolic syndrome: obesity, insulin resistance, dyslipidemia and hypertension. For clinical practice, the International Diabetes Federation has made the following definition: Central obesity, defined as waist circumference ≥94 cm for Europid men and ≥80 cm for Europid women, with ethnicity specific values for other groups, plus any two of the following four factors:
raised triglycerides (TG) level: ≥150 mg/dL (1.7 mmol/L);
reduced HDL cholesterol: <40 mg/dL in males and <50 mg/dL in females;
raised blood pressure (BP): systolic BP≥130 or diastolic BP≥85 mm Hg;
raised fasting plasma glucose (FPG)≥100 mg/dL (5.6 mmol/L).

The presence of metabolic syndrome causes an increased risk on diabetes type II and cardiovascular diseases. Prevalence cannot be given precisely, as it is dependent on the definition, region, gender, and age. In a study in the United States, 26% of the adults (>20 years) had impaired fasting glucose levels. The prevalence of impaired fasting glucose levels increased with age, peaking at 39% in the 65+ population. In Europe, the percentages differ between 5% to 37% for adults between 40 and 55 years.

### Role for the Microbiota in Metabolic Syndrome

A number of studies have shown that specific relationships exist between intestinal microbiota and human metabolism. Young conventionally reared mice have 42% more total body fat than germ-free mice, although their energy intake was lower. Colonization of young germ-free mice with microbiota from conventionally reared mice produced a 60% increase in body fat mass that is associated with increased insulin resistance, despite lower energy intake. Feces transplantation with microbiota from obese mice resulted in a significantly greater increase in total body fat than colonization with microbiota from lean donors. Obesity has been associated with phylum-level changes in the composition of the intestinal microbiota. Increase in the relative abundance of Firmicutes and reduction in the level of Bacteroidetes has been observed in intestinal microbiota of obese mice (ob/ob) and humans. However, a number of other more recent studies have shown variable results with respect to the compositional changes in the microbiota of obese human individuals. Although the relationships between the microbiota and development of obesity and metabolic syndrome is still not completely elucidated, there are already described several (possible) mechanisms by which the gut microbiota can influence host metabolism.

Since the intestinal barrier function plays a crucial role in these, this rationale will elucidate this mechanism of action and the influence of probiotics on that.

The intestinal mucosa is the largest interface between the outside world and the human internal milieu. Across a surface area that approximates the size of a soccer field, it is here where the highest concentration of bacteria is prevented from invading our internal environment while allowing nutrient and water absorption by a single cell layer of epithelium. The ability to control the invasion of harmful content from the lumen is called intestinal mucosal barrier function. While the epithelial layer forms the most obvious physical boundary between inner and outer environment, the full complexity of factors that control intestinal barrier function reaches beyond the epithelium and is not fully understood. Throughout the intestine a single layer of epithelial cells covers the inner surface and is responsible for this barrier function. Tight junctions are protein structures that allow selective passage of ions and small molecules, but form, in healthy subjects, a tight barrier to protein seized molecules and bacteria. To make matters more complicated, the task of the epithelium is not only to keep bacteria and antigens out while absorbing nutrients, but also to allow contact between luminal contents and immune cells. This occurs through limited and highly controlled uptake of antigens and bacteria. This seemingly paradoxical task is, however, crucial in the induction of targeted and protective mucosal immune responses to pathogens as well as to the development of oral tolerance to commensals and food antigens.

The barrier function of the intestine can be influenced by different factors, like heredity, bacterial flora, diet, psychological stress, oxidative stress, exercise, and drugs. Increased permeability of the epithelial barrier has been associated with many gastrointestinal inflammatory disorders, like inflammatory bowel diseases (Crohn's disease, ulcerative colitis and pouchitis), celiac disease. An increased permeability can also lead to increased levels of endotoxins in the blood, which are linked to systemic inflammatory diseases, like metabolic syndrome, diabetes, atherosclerosis, chronic fatigue syndrome, autism, migraine and rheumatoid arthritis.

Leakage of LPS from the Lumen to the Body

The increased permeability leads to increased levels of endotoxins, specifically lipopolysaccharides (LPS). The human gut serves as reservoir of LPS, which is the major component of the outer membrane of Gram-negative bacteria. Several studies have demonstrated that low levels of LPS are detectable in the blood of healthy humans, suggesting that LPS is continuously absorbed at a low rate from the gut. Interestingly, circulating LPS correlated with insulin levels and patients with type 2 diabetes have increased amounts of circulating LPS. In addition, endotoxemia levels were associated with an increased risk of diabetes, suggesting that prevention of LPS leakage can also have a preventive effect. In an experiment where human volunteers got intravenous LPS, systemic insulin resistance was induced. Two mechanisms of LPS absorption from the gut to the circulatory system have been proposed: chylomicron-facilitated transport and extracellular leakage through tight junctions in the epithelial lining.

Tight junction proteins are important players in the epithelial barrier function and play a role in the paracellular leakage of LPS. Some probiotic bacteria have shown to improve the expression of these proteins in Caco-2 cell lines and in vivo, indicating that enhancing the expression of genes involved in tight junction signaling is a possible mechanism by which bacteria can improve intestinal barrier function.

### Autism Spectrum Disorders

Gastrointestinal (GI) dysfunction has been reported in a substantial number of children with autism spectrum disorders (ASD). Activation of the mucosal immune response and the presence of abnormal gut microbiota are repeatedly observed in these children. In children with ASD, the presence of GI dysfunction is often associated with increased irritability, tantrums, aggressive behavior, and sleep disturbances. Moreover, modulating gut bacteria with short-term antibiotic treatment can lead to temporary improvement in behavioral symptoms in some individuals with ASD. Probiotics can influence microbiota composition and intestinal barrier function and alter mucosal immune responses. The administration of a probiotic composition according to the disclosure as provided herein to address changes in the microbiota might, is a useful novel therapeutic tool with which to restore normal gut microbiota, reduce inflammation, restore epithelial barrier function, and ameliorate behavioral symptoms associated with children with ASD.

### Probiotics and Metabolic Syndrome

Currently, there is strong support for the use of probiotics in the clinical prevention or treatment of metabolic syndrome. First of all, there are animal models reporting beneficial effects by various strains of Lactobacilli on characteristics of type 2 diabetes. A more recent study showed translocation of intestinal bacteria to adipose tissue and blood after a high-fed diet. This translocation was dependent on Nod1 or CD14 and suppressed by Myd88. Bacterial translocation could be reversed by treatment with the probiotic strain Bifidobacterium animalis subsp. lactis 420, which improves the animals' overall inflammatory and metabolic status. So far, some human studies have been performed with probiotics interventions measuring different parameters involved in lipid metabolism, metabolic syndrome or type 2 diabetes.

### Inhibition of Pro-Inflammatory Cytokines

Obesity induced by high-fat diets and the associated metabolic disorders are characterized by a state of low-grade inflammation. The inflammation has been related to alterations in the gut microbiota composition and increased plasma LPS levels. Probiotics can work on both two levels; they can decrease the leakage of LPS as shown in the paragraphs before, or they can decrease the inflammation more directly. A large part of the immune system (approximately 80%) is concentrated in and around the intestinal mucosa. The intestinal microbiota is involved in maturation of the immune system as demonstrated in studies in germ-free mice. In turn, the microbiota in the intestine plays an important role in the regulation of functions in the immune system. The immune system can be modulated by probiotic bacteria and these effects are highly species- and strain-specific. Inflammatory signals cause disruption of the epithelial barrier. Interleukin-10 (IL-10) is an important cytokine to regulated immune responses and to prevent excessive pro-inflammatory responses. Induction of IL-10 by different probiotic strains was measured with an in vitro test with peripheral blood mononuclear cells.

The incidence of obesity, metabolic syndrome, and type 2 diabetes is growing. The intestinal microbiota and the epithelial barrier function might play an important role in these conditions. Probiotic bacteria can have an effect on the epithelial barrier via different molecular pathways and routes. Therefore, a multispecies product is a very good choice to support these different routes. All compositions provided in the application were carefully selected based on their in vitro capacities to improve the epithelial barrier function and have positive effects on systemic inflammatory diseases, like metabolic syndrome, atherosclerosis and diabetes type II.

### Migraine

Migraine prevalence is associated with gastrointestinal disorders. Possible underlying mechanisms could be increased gut permeability and inflammation. Probiotics may decrease intestinal permeability as well as inflammation and, therefore, may reduce the frequency and/or intensity of migraine attacks. Therefore, feasibility, possible clinical efficacy, and adverse reactions of probiotic treatment in migraine patients were assessed. Twenty-nine migraine patients took 2 g/d of a probiotic food supplement (ECOLOGIC® Barrier, 2.5×10⁹ cfu/g) during twelve weeks. Participants recorded frequency and intensity of migraine in a headache diary and completed the Migraine Disability Assessment Scale (MIDAS) and Henry Ford Hospital Headache Disability Inventory (HDI) at baseline and after twelve weeks of treatment. Compliance was measured every four weeks by counting the remaining sachets with probiotics. The study was completed by 27/29 (93%) patients who took 95% of the supplements. Obstipation was reported by four patients during the first two weeks of treatment only. The mean±standard deviation (SD) number of migraine days/month decreased significantly from 6.7±2.4 at baseline to 5.1±2.2 (P=0.008) in week 5-8 and 5.2±2.4 in week 9-12 (P=0.001). The mean±SD intensity of migraine decreased significantly from 6.3±1.5 at baseline to 5.5±1.9 after treatment (P=0.005). The MIDAS score improved from 24.8±25.5 to 16.6±13.5 (P=0.031). However, the mean HDI did not change significantly. In conclusion, probiotics may decrease migraine supporting a possible role for the intestine in migraine management. Feasibility and lack of adverse reactions justify further placebo-controlled studies.

### Kidney Disorders

### Kidney Disease and Kidney Transplantation

The intestinal epithelium forms a barrier between the intestinal lumen and systemic circulation. The ability to control the translocation of bacteria, toxins and antigens from the lumen to the circulation is termed as intestinal barrier function. The intestinal barrier can be disturbed by dysbiotic gut microbiota resulting from uremia in chronic kidney disease (CKD), obesity, high-fat diet, and high-fructose diet. This can lead to an increased intestinal permeability, permitting the entrance of lipopolysaccharides (LPS) into the circulation. LPS, also known as endotoxin, are found in the outer membrane of the cell wall of gram-negative bacteria that reside in the intestinal lumen as part of gut microbiota. Upon translocation into the circulation, LPS trigger the release of pro-inflammatory cytokines, resulting in systemic inflammation and oxidative stress. Consequently, increased intestinal permeability results in systemic inflammatory conditions manifesting as atherosclerosis, depression or chronic fatigue syndrome.

Endotoxemia and systemic inflammation are significant independent predictors of fatigue, depression and endothelial dysfunction in chronic kidney disease and kidney transplant recipients (KTRs). Fatigue and depression are highly common among KTRs, occurring in up to 59% and 41% of these patients respectively, and substantially impacting on quality of life (QoL). Depression is known to be associated with non-adherence to immunosuppressive medication, graft loss, and all-cause mortality. More importantly, endothelial dysfunction, an early marker of atherosclerosis, contributes to increased risk of cardiovascular disease, the leading cause of graft loss and mortality in KTRs.

Probiotic therapy with a composition as provided herein also improves intestinal barrier function in KTRs, reducing endotoxemia and systemic inflammation, translating as improved fatigue, depression and endothelial function.

TABLE 1
Mean pre- and post-intervention scores and standard error of the means
(shown in parentheses) on the LEIDS-R, BDI and BAI in the Placebo
and Probiotics groups. Asterisks indicate significant treatment effect
differences between pre- and post-intervention assessments.

| | | (Pre-intervention) | | (Post-intervention) | |
|---|---|---|---|---|---|
| LEIDS-R | | | | | |
| Aggression | Placebo | 8.80 | (0.94) | 8.45 | (0.98) |
| | Probiotics** | 8.68 | (0.94) | 6.25 | (0.98) |
| Control | Placebo | 7.65 | (0.80) | 6.70 | (0.82) |
| | Probiotics | 7.25 | (0.83) | 5.80 | (0.82) |
| Hopelessness | Placebo | 5.60 | (0.85) | 4.70 | (0.74) |
| | Probiotics | 4.75 | (0.85) | 4.0 | (0.74) |
| Risk Aversion | Placebo | 9.50 | (0.93) | 9.25 | (0.87) |
| | Probiotics | 10.00 | (0.93) | 7.95 | (0.87) |
| Rumination | Placebo | 11.75 | (0.90) | 11.85 | (0.93) |
| | Probiotics*** | 11.20 | (0.90) | 8.25 | (0.93) |
| Acceptance | Placebo | 1.40 | (0.34) | 1.35 | (0.37) |
| | Probiotics | 0.90 | (0.34) | 1.10 | (0.37) |
| Total | Placebo | 44.70 | (3.24) | 42.30 | (3.51) |
| | Probiotics*** | 42.75 | (3.24) | 33.35 | (3.51) |
| BDI | | | | | |
| Affective | Placebo | 2.45 | (0.39) | 2.30 | (0.43) |
| | Probiotics | 2.10 | (0.39) | 2.00 | (0.43) |
| Somatic | Placebo | 6.65 | (0.84) | 6.80 | (0.88) |
| | Probiotics | 5.85 | (0.84) | 5.25 | (0.88) |
| Total | Placebo | 9.10 | (1.00) | 9.10 | (1.19) |
| | Probiotics | 7.90 | (1.00) | 7.25 | (1.19) |
| | Placebo | 12.21 | (1.70) | 11.21 | (1.69) |
| | Probiotics | 11.35 | (1.66) | 9.95 | (1.65) |

*p < .05,
**p < .01,
***p < .001.

TABLE 2
Overview of in vitro results of individual strains present in ECOLOGIC ® Barrier.

| | Strengthening | Strengthening | Inhibition | Inhibition pro- | |
|---|---|---|---|---|---|
| | epithelial barrier | epithelial barrier | mast cell | inflammatory | Decrease |
| Strain | (TNF-α & IL1-β) | (Salmonella) | activation | cytokines | LPS load |
| B. bifidum W23 | +++++++ | | +++++++ | +++++++ | +++++++ |
| B. lactis W52 | | +++++++ | +++++++ | +++++++ | |
| L. acidophilus W37 | +++++++ | | | +++++++ | +++++++ |
| L. brevis W63 | +++++++ | | | +++++++ | |
| L. casei W56 | +++++++ | +++++++ | +++++++ | +++++++ | |
| L. salivarius W24 | | | +++++++ | | |
| Lc. lactis W19 | +++++++ | | | | +++++++ |
| Lc. lactis W58 | | +++++++ | | +++++++ | |

Cells with +++++++ = positive effect;
Blank cells = no data or no effect.

### REFERENCES

van der Does W., J. M. G. Williams (2003). Leiden Index of Depression Sensitivity-Revised (LEIDS-R). Leiden University.
van der Does W. (2005). Thought suppression and cognitive vulnerability to depression. Brit. J. Clin. Psychol. 44:1-14. doi: 10.1348/014466504x19442
Beck A. T., R. A. Steer, R. Bal, and W. F. Ranieri (1996). Comparison of Beck Depression Inventories-IA and-II in psychiatric outpatients. J. Pers. Assess. 67:588-597. doi: 10.1207/s15327752jpa6703_13
Beck A. T., N. Epstein, G. Brown, and R. A. Steer (1988). An inventory for measuring clinical anxiety: psychometric properties. J. Consult. Clin. Psych. 56:893. doi: 10.1037//0022-006x.56.6.893
Sheehan D. V., Y. Lecrubier, K. H. Sheehan, P. Amorim, and J. Janays, et al. (1998). The Mini-International Neuropsychiatric Interview (MINI): The development and validation of a structured diagnostic psychiatric interview for DSM-IV and ICD-10. J. Clin. Psychiat. 59:22-33.
Elzinga B. M., A. M. Ardon, M. K. Heijnis, M. B. De Ruiter, R. Van Dyck, et al. (2007). Neural correlates of enhanced working-memory performance in dissociative disorder: a functional MM study. Psychol. Med. 37:235-246. doi: 10.1017/s0033291706008932
van der Does W. (2002b). Handleiding bij de Nederlandse versie van Beck Depression Inventory (BDI-II-NL). Amsterdam: Pearson.
Dozois D. J., K. S. Dobson, and J. L. Ahnberg (1998). A psychometric evaluation of the Beck Depression Inventory-II. Psychol. Assess. 10:83. doi: 10.1037/1040-3590.10.2.83
van der Does W. (2002a). Cognitive reactivity to sad mood: structure and validity of a new measure. Behav. Res. Ther. 401:105-120. doi: 10.1016/s0005-7967(00)00111-x
Bouman T. K. (1994). De Beck Depression Inventory (BDI). Gedragstherapie 27:69-72.
Masson M. E. J. (2011). A tutorial on a practical Bayesian alternative to Null Hypothesis Significance Testing. Behav. Res. Meth. 43:679-690. doi: 10.3758/s13428-010-0049-5
Wagenmakers E.-J. (2007). A practical solution to the pervasive problems of p values. Psychon. B. Rev. 14:779-804. doi: 10.3758/bf03194105
Raftery A. E. (1995). Bayesian model selection in social research. In: Marsden PV editor. Sociological methodology, Oxford, U. K.: Blackwells. pp. 111-196. doi: 10.2307/271063
Kuehner C., and I. Weber (1999). Responses to depression in unipolar depressed patients: An investigation of Nolen-Hoeksema's response styles theory. Psychol. Med. 29:1323-1333.
Spasojevic J., and L. B. Alloy (2001). Rumination as a common mechanism relating depressive risk factors to depression. Emotion 1:25-37.
Nemeroff C. B., H. S. Mayberg, S. E. Krahl, J. McNamara, and A. Frazer, et al. (2006). VNS therapy in treatment-resistant depression: clinical evidence and putative neurobiological mechanisms. Neuropsychopharmacol. 31:1345-1355. doi: 10.1038/sj.npp.1301082
Vonck K., R. Raedt, J. Naulaerts, F. De Vogelaere, and E. Thiery, et al. (2014). Vagus nerve stimulation . . . 25 years later! What do we know about the effects on cognition? Neurosci. Biobehav. Rev. 45:63-71. doi: 10.1016/j.neubiorev.2014.05.005
Grimm S., and M. Bajbouj (2010). Efficacy of vagus nerve stimulation in the treatment of depression. Expert Rev. Neurother. 10:87-92. doi: 10.1586/ern.09.138
Schultz A. L. (1987) 5-Hydroxyindoleacetic Acid. In: A. J. Pesce and L. A. Kaplan, editors. Methods in Clinical Chemistry, St. Louis, Mo.: Mosby-Year Book Inc. pp. 714-720.
Teixeira T. F. S., A. P. B. Moreira, N. C. S. Souza, R. Frias, and M. D. C. G. Peluzio (2014). Intestinal permeability measurements: general aspects and possible pitfalls. Nutr. Hosp. 2:269-281.

1-42. (canceled)

43. A multispecies probiotic composition comprising:
Bifidobacterium bifidum W23, Lactobacillus acidophilus W37, Lactobacillus brevis W63, Lactobacillus casei W56, Lactobacillus salivarius W24, Bifidobacterium lactis W52, Lactococcus lactis W19, and Lactococcus lactis W58 formulated to treat or prevent depression in a human.

44. The multispecies probiotic composition according to claim 43, further formulated to treat or prevent rumination in the human.

45. The multispecies probiotic composition according to claim 43, further formulated to treat or prevent aggression in the human.

46. A method for treating or preventing depression in a human subject, the method comprising:
administering to the human subject the multispecies probiotic composition of claim 43.

47. A method for treating or preventing rumination in a human subject, the method comprising:
administering to the human subject the multispecies probiotic composition of claim 44.

48. A method for treating or preventing aggression in a human subject, the method comprising:
administering to the human subject the multispecies probiotic composition of claim 45.

49. The method according to claim 46, additionally comprising treating rumination in the human subject.

50. The method according to claim 46, additionally comprising treating aggression in the human subject.

51. The method according to claim 46, further comprising providing the human subject with the multispecies probiotic composition for at least four weeks.

52. The method according to claim 46, wherein the human subject has a daily intake of the multispecies probiotic composition of at least 2×10⁸ cfu (colony-forming units).

53. The method according to claim 46, wherein the human subject has a daily intake of the multispecies probiotic composition of at least 2×10⁹ cfu (colony-forming units).

54. The method according to claim 47, wherein the human subject has a daily intake of the multispecies probiotic composition of at least 2×10⁸ cfu (colony-forming units).

55. The method according to claim 47, wherein the human subject has a daily intake of the multispecies probiotic composition of at least 2×10⁹ cfu (colony-forming units).

56. The method according to claim 48, wherein the human subject has a daily intake of the multispecies probiotic composition of at least 2×10⁸ cfu (colony-forming units).

57. The method according to claim 48, wherein the human subject has a daily intake of the multispecies probiotic composition of at least 2×10⁹ cfu (colony-forming units).

58. The method according to claim 49, wherein the human subject has a daily intake of the multispecies probiotic composition of at least 2×10⁸ cfu (colony-forming units).

59. The method according to claim 49, wherein the human subject has a daily intake of the multispecies probiotic composition of at least 2×10⁹ cfu (colony-forming units).

60. The method according to claim 50, wherein the human subject has a daily intake of the multispecies probiotic composition of at least 2×10⁸ cfu (colony-forming units).

61. The method according to claim 50, wherein the human subject has a daily intake of the multispecies probiotic composition of at least 2×10⁹ cfu (colony-forming units).

62. The method according to claim 51, wherein the human subject has a daily intake of the multispecies probiotic composition of at least 2×10⁸ cfu (colony-forming units).
